(19) United States
(12) Patent Application Publication
(10) Pub. No.: US 2023/0111680 A1
(43) Pub. Date: Apr. 13, 2023

(54) PROSTHETIC IMPLANTS FOR DISPLACING LEAFLETS

(71) Applicant: Edwards Lifesciences Corporation, Irvine, CA (US)

(72) Inventors: Joseph Mordechai Leichner, Netanya (IL); Hanoch Cohen-Tzemach, Holon (IL); Jeong Soo Lee, Diamond Bar, CA (US)

(21) Appl. No.: 18/065,147

(22) Filed: Dec. 13, 2022

Related U.S. Application Data
(63) Continuation of application No. PCT/US2021/037474, filed on Jun. 15, 2021.
(60) Provisional application No. 63/040,235, filed on Jun. 17, 2020.

(51) Int. Cl. A61F 2/24

(52) U.S. Cl. CPC A61F 2/2418; A61F 2/2427; A61F 2220/0075; A61F 2250/001; A61F 2/9522

(57) ABSTRACT

Embodiments disclosed herein may be directed to devices, systems, and methods for addressing leaflets within a patient's body, including displacement of such leaflets. The leaflets may be of a native heart valve, or may be of a prosthetic heart valve that has been previously implanted within the patient's body. The leaflets may be displaced to reduce the possibility of the leaflets blocking access to structures within the patient's body, which may comprise cardiac structures such as coronary ostia, for example. As such, a reduced possibility of maladies caused by blockage of the cardiac structures may result.

### RELATED APPLICATION

This application is a continuation of PCT patent application no. PCT/US2021/037474, filed Jun. 15, 2021, which application claims the benefit of, and priority to, U.S. Provisional Patent Application Ser. No. 63/040,235 filed on Jun. 17, 2020, the specification of each of these applications being incorporated herein in its entirety by this specific reference.

### BACKGROUND OF THE INVENTION

A variety of maladies may affect an individual's body. Such maladies may be of the individual's heart, and may include maladies of the individual's heart valves, including the aortic, mitral, tricuspid, and pulmonary valves. Stenosis, for example, is a common and serious valve disease that may affect the operation of the heart valves and an individual's overall well-being.

Implants may be provided that may replace or repair portions of a patient's heart. Prosthetic implants, such as prosthetic valves, may be provided to replace a portion of a patient's heart. Prosthetic aortic, mitral, tricuspid, and even pulmonary valves may be provided.

Implants may be deployed to the desired portion of the patient's body percutaneously, in a minimally invasive manner. Such deployment may occur transcatheter, in which a catheter may be deployed through the vasculature of an individual.

During deployment of such implants to a native heart valve for example, the native heart valve leaflets may remain within the patient's body. The native leaflets may be pushed aside by the deployment of the prosthetic valve, with the prosthetic leaflets performing the function previously provided by the native leaflets. The native leaflets that remain in the body though, may be undesirably positioned, as they may block access to cardiac structures. Such structures may include one or more coronary ostia, for example. Blocking access to such structures may result in other health conditions that may be undesirable. Improvements accordingly may be desired in addressing positions of native structures within a patient's body, including native leaflets.

### SUMMARY

Embodiments disclosed herein may be directed to devices, systems, and methods for addressing leaflets within a patient's body, including displacement of such leaflets. The leaflets may be of a native heart valve, or may be of a prosthetic heart valve that has been previously implanted within the patient's body. The leaflets may be displaced to reduce the possibility of the leaflets blocking access to structures within the patient's body, which may comprise cardiac structures such as coronary ostia, for example. As such, a reduced possibility of maladies caused by blockage of the cardiac structures may result. Further, access to the cardiac structures following implantation may be made easier by the leaflets not blocking access to such structures.

Embodiments disclosed herein may include a prosthetic valve. The prosthetic valve may include a valve body having a proximal end, a distal end, an outer surface, and an inner surface facing a flow channel. A plurality of valve leaflets may be positioned within the flow channel and may extend inward from the inner surface of the valve body. One or more protrusions may be configured to extend outward from the outer surface of the valve body and configured to displace one or more valve leaflets positioned outside of the flow channel of the valve body in a distal direction.

Embodiments disclosed herein may include a method. The method may include expanding a prosthetic valve within a patient's body, the prosthetic valve including a valve body having a proximal end, a distal end, an outer surface, and an inner surface facing a flow channel, and a plurality of valve leaflets positioned within the flow channel and extending inward from the inner surface of the valve body. The method may include displacing one or more valve leaflets positioned outside of the flow channel of the valve body in a distal direction with one or more protrusions extending outward from the outer surface of the valve body.

Embodiments disclosed herein may include a crimping device for a prosthetic valve. The crimping device may include a compressive body having an inner surface surrounding a channel configured to receive the prosthetic valve, the inner surface configured to be contracted to apply a compressive force to the prosthetic valve within the channel to crimp the prosthetic valve, the inner surface having a recess shaped in the inner surface to accommodate a protrusion of the prosthetic valve. The crimping device may include an actuator for contracting the inner surface.

Embodiments disclosed herein may include a method for crimping a prosthetic valve. The method may include positioning the prosthetic valve within a channel of a crimping device having an inner surface surrounding the channel. The method may include positioning a protrusion of the prosthetic valve within a recess shaped in the inner surface to accommodate the protrusion of the prosthetic valve. The method may include compressing the inner surface against the prosthetic valve to crimp the prosthetic valve.

### BRIEF DESCRIPTION OF THE DRAWINGS

These and other features, aspects, and advantages are described below with reference to the drawings, which are intended to illustrate, but not to limit, the disclosure. In the drawings, like reference characters denote corresponding features consistently throughout similar embodiments.

FIG. 1 is a perspective view of a prosthetic valve according to an embodiment of the present disclosure.

FIG. 2 is top schematic view of the prosthetic valve shown in FIG. 1.

FIG. 3 is a cross sectional schematic view of the prosthetic valve shown in FIG. 1.

FIG. 4 is a cross sectional schematic view of the prosthetic valve shown in FIG. 1, in an unexpanded configuration and within an aortic annulus of a patient's body.

FIG. 5 is a cross sectional schematic view of the prosthetic valve shown in FIG. 1, in an expanded configuration and within an aortic annulus of a patient's body.

FIG. 6 is a cross sectional schematic view of a prosthetic valve according to an embodiment of the present disclosure.

FIG. 7 is a plan view of a frame of a prosthetic valve in a flattened configuration according to an embodiment of the present disclosure.

FIG. 8 is a plan view of a frame of a prosthetic valve in a flattened configuration according to an embodiment of the present disclosure.

FIG. 9 is a cross sectional schematic view of a prosthetic valve including the frame shown in FIG. 8 according to an embodiment of the present disclosure.

FIG. 10 is a perspective view of a prosthetic valve according to an embodiment of the present disclosure.

FIG. 11 is a plan view of a frame of a prosthetic valve in a flattened configuration according to an embodiment of the present disclosure.

FIG. 12 is a plan view of a frame of a prosthetic valve in a flattened configuration according to an embodiment of the present disclosure.

FIG. 13 is a cross sectional schematic view of a prosthetic valve including the frame shown in FIG. 12 according to an embodiment of the present disclosure.

FIG. 14 is a plan view of a frame of a prosthetic valve in a flattened configuration according to an embodiment of the present disclosure.

FIG. 15 is a close up view of a portion of the frame shown in FIG. 14.

FIG. 16 is a cross sectional schematic view of a prosthetic valve including the frame shown in FIG. 14 according to an embodiment of the present disclosure.

FIG. 17 is a cross sectional schematic view of the prosthetic valve shown in FIG. 16 with protrusions extending radially outward.

FIG. 18 is a side schematic view of a prosthetic valve according to an embodiment of the present disclosure.

FIG. 19 is a side schematic view of the prosthetic valve shown in FIG. 18 with the prosthetic valve expanded and protrusions extending radially outward.

FIG. 20 is a cross sectional schematic view of the prosthetic valve shown in FIG. 19 with protrusions extending radially outward.

FIG. 21 is a cross sectional schematic view of a prosthetic valve according to an embodiment of the present disclosure with protrusions extending radially outward.

FIG. 22 is a cross sectional schematic view of a prosthetic valve according to an embodiment of the present disclosure.

FIG. 23 is a perspective view of a prosthetic valve according to an embodiment of the present disclosure.

FIG. 24 is a perspective view of a crimping device according to an embodiment of the present disclosure.

FIG. 25 is a cross sectional schematic view of a channel of the crimping device shown in FIG. 24.

FIG. 26 is a cross sectional schematic view of the channel of the crimping device shown in FIG. 25 with a prosthetic valve and elongate shaft of a delivery system positioned within the channel.

FIG. 27 is a cross sectional schematic view of the channel of the crimping device shown in FIG. 25 with a prosthetic valve crimped within the channel.

FIG. 28 is a side view of a delivery apparatus according to an embodiment of the present disclosure.

### DETAILED DESCRIPTION

The following description and examples illustrate some example embodiments of the disclosure in detail. Those of skill in the art will recognize that there are numerous variations and modifications of the disclosure that are encompassed by its scope. Accordingly, the description of a certain example embodiment should not be deemed to limit the scope of the present disclosure.

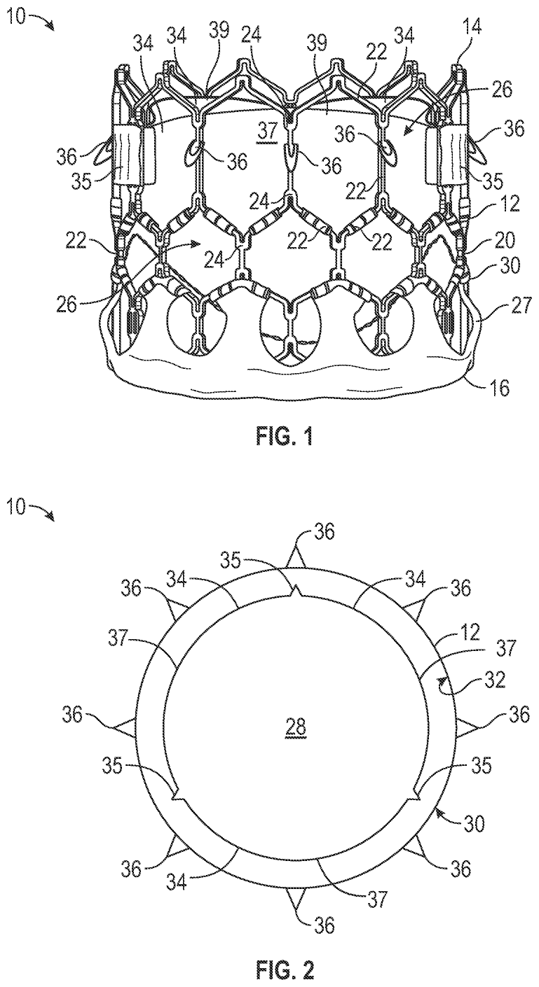

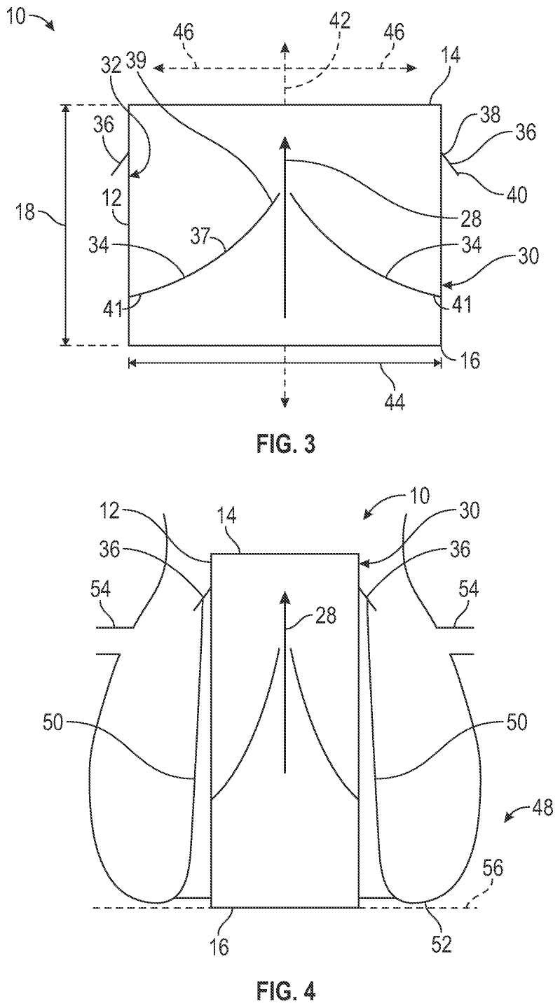

FIG. 1 illustrates a perspective view of an implant in the form of a prosthetic valve 10. The prosthetic valve 10 may include a valve body 12 that may have a proximal end 14, a distal end 16, and a length 18 (as marked in FIG. 3) between the proximal end 14 and the distal end 16. The proximal end 14 may comprise an outflow end of the prosthetic valve 10, and the distal end 16 may comprise an inflow end of the prosthetic valve 10.

The valve body 12 may include a frame 20 that may include a plurality of struts 22 that may join together at junctures 24 and may have spaces 26 between the struts 22. The spaces 26 may comprise openings of the frame 20 that may allow fluid flow therethrough or the passage of other components therethrough. The configuration of the frame may vary in other embodiments.

The frame 20 may be configured to allow the valve body 12 to be collapsible and expandable, with the frame 20 being crimped to move to a collapsed (or undeployed or unexpanded) state and being expanded to move to an expanded (or deployed) state. The plurality of struts 22 may be configured to move closer together to allow the frame 20 to move to the collapsed state. The width of the openings between the struts 22 may be reduced as the frame 20 is moved to the collapsed (or undeployed or unexpanded) state, with the length of the openings increasing. The struts 22 of the frame 20 may be configured to circumferentially move away from each other to move to the expanded state. The width of the openings between the struts 22 may be increased as the frame 20 is moved to the expanded (or deployed) state, with the length of the openings decreasing.

The valve body 12 may further include one or more covers 27 that may cover a portion of the frame 20. A cover 27 may extend circumferentially about the frame 20, and may be positioned to form all or a portion of an outer surface 30 of the valve body 12. A cover 27 may enhance securement of the deployed prosthetic valve 10 to a desired location within the patient's body.

The valve body 12 may surround a flow channel 28 (as marked in FIGS. 2 and 3) that may allow for flow of fluid (e.g., blood or another fluid) through the valve body 12. The valve body 12 may include an outer surface 30 that may face outward from the valve body 12 and may include an inner surface 32 (as marked in FIGS. 2 and 3) that faces the flow channel 28. The outer surface 30 may comprise an anchoring surface that may be utilized to anchor the prosthetic valve 10 within the desired portion of the patient's body (e.g., a heart valve annulus if desired). The outer surface 30 may apply a force radially outward to anchor the prosthetic valve 10 within an annulus.

A plurality of valve leaflets 34 may be positioned within the flow channel 28 and may extend inward from the inner surface 32 of the valve body 12. The valve leaflets 34 may include outer end portions 35 (marked in FIGS. 1 and 2) that couple to the frame 20 of the valve body 12 and may include inner portions 37 that are drawn towards each other when the valve leaflets 34 are closed. The outer end portions 35 may couple to the frame 20 at commissure points of the leaflets 34 and may pass through openings of the frame 20 to couple to the frame 20. Each leaflet 34 may include a proximal end 39 (as marked in FIG. 3) that may form an edge of the leaflet 34 in the outflow direction. The proximal end 39 may be opposite the distal ends 41 of each leaflet (as marked in FIG. 3). The prosthetic valve 10 may include three leaflets 34 as shown in FIGS. 1 and 2, or may include a greater or lesser number of leaflets 34 as desired.

The valve leaflets 34 may move between an opened state (in which fluid flows through the flow channel 28) and a closed state (in which fluid flow is impeded through the flow channel 28), which may mimic the motion of native heart valve leaflets. The valve leaflets 34 may have the proximal ends 39 move towards each other in a radially inward manner and contact each other to close the valve, and then move away from each other in a radially outward manner to open the valve. The valve leaflets 34 may open in a proximal direction. FIG. 2, for example, illustrates the valve leaflets 34 in an opened state, with the valve leaflets 34 positioned away from each other in a radially outward manner.

The plurality of valve leaflets 34 may be configured to open to allow for flow in the proximal or outflow direction of the prosthetic valve 10, and may be configured to close to impede flow in the distal or inflow direction of the prosthetic valve 10.

Referring to FIG. 1, the prosthetic valve 10 may include one or more protrusions 36 that are configured to extend outward from the outer surface 30 of the valve body 12. The one or more protrusions 36 may be configured to displace one or more valve leaflets positioned outside of the flow channel 28 of the valve body 12 in a distal direction. The one or more protrusions 36 may each have a first end 38 (as marked in FIG. 3) coupled to the frame 20 of the valve body 12 and may extend outward to a second end 40 of the protrusion 36. The second end 40 of the protrusion 36 may comprise a tip of the protrusion 36 that may be atraumatic. Each protrusion 36, as shown in FIGS. 1 and 3, may be angled towards a distal direction, with the second end 40 positioned more distal than the first end 38. Each protrusion 36 may have a linear shape, as shown, or may have another shape as desired. Each protrusion 36 may comprise a prong in embodiments.

Each protrusion 36 may be positioned axially upon the valve body 12 at a position that may allow the protrusion 36 to contact a native (or host) leaflet upon expansion of the valve body 12. Each protrusion 36 may be positioned in a variety of locations, and may be positioned on a proximal portion of the valve body 12 (as shown in FIG. 1). The protrusions 36 may be positioned between the proximal end 14 and distal end 16 of the valve body 12, although other locations may be utilized as desired. In embodiments, the protrusions 36 may be axially positioned distal of the proximal ends 39 of the prosthetic leaflets 34 (when in such leaflets 34 are in an opened state, and/or when such leaflets 34 are in a closed state). Such positioning may allow the protrusions 36 to displace the native (or host) leaflet if the native or host leaflet has a proximal end at the same axial position as the proximal ends 39 of the prosthetic leaflets 34. As such, the protrusions 36 may be axially positioned closer to the inflow or distal end 16 of the valve body 12 than the proximal ends of the native or host leaflets. In other embodiments, other positions of the protrusions 36 may be utilized.

Each protrusion 36 may be spaced from each other circumferentially about the outer surface 30 of the valve body 12. FIG. 2, for example illustrates a top schematic view of the prosthetic valve 10 showing the protrusions 36 extending radially outward from the outer surface 30 and circumferentially spaced from each other about the outer surface 30 of the valve body 12. The spacing may be equal as shown in FIG. 2, or other varied spacing may be utilized in embodiments as desired.

In embodiments, the number of protrusions 36 may be eight as shown in FIG. 2, or a greater or lesser number may be provided as desired. For example, in one embodiment, three protrusions 36 may be utilized, with one for each native or host leaflet to be displaced. In one embodiment, only one protrusion may be utilized. In one embodiment, two may be utilized, or a greater number (e.g., four or greater) may be utilized. An increased number of protrusions 36 may enhance the likelihood of the protrusions 36 displacing a native or host leaflet. The number of protrusions 36 may be selected to match the number of leaflets to be displaced, or may be provided as a different number of protrusions 36.

FIG. 2 illustrates the valve leaflets 34 in an opened configuration, allowing fluid flow through the flow channel 28.

The valve body 12 may have a cylindrical shape as shown in FIGS. 1 and 2, or may have another shape (e.g., a tapered or “V” shape, or a bulb shape, or other desired shape). A cylindrical shape may include a uniform outer diameter for the outer surface 30 of the valve body 12 in embodiments, and may include a uniform inner diameter of the inner surface 32. Other configurations of cylindrical shapes may be utilized as desired.

FIG. 3 illustrates a cross sectional schematic view of the prosthetic valve 10. The valve body 12 extends along and surrounds an axis 42 of the valve body 12. The valve body 12 has a width 44 transverse to the axis 42 of the valve body 12. The valve body 12 may have a length 18 that varies in response to a variation in the width 44 of the valve body 12. For example, referring back to FIG. 1, each space 26 between the struts 22 of the frame 20 may have a width and a length. The valve body 12 may be collapsible and expandable, and in the collapsed state the struts 22 may be drawn together such that the width of each space 26 is less than shown in FIG. 1, and the length of each space 26 is greater than shown in FIG. 1. As the valve is expanded radially outward (as marked with arrows 46 in FIG. 3) from the axis 42, the length of each space 26 may decrease and the width of each space 26 may increase. The corresponding change of size of each space and the movement of the struts 22 may allow the entire frame 20 to have its length decrease and its width increase.

As such, upon expansion of the valve body 12, the width 44 of the valve body 12 may increase, which correspondingly decreases the length 18 of the valve body 12. The expansion may be controlled such that a proximal end 14 or distal end 16 of the valve body 12 may be held in position such that the decrease in length 18 occurs in a direction. For example, if the distal end 16 is held in position and the valve body 12 is expanded, then the proximal end 14 may move towards the distal end 16. If the proximal end 14 were held in position then the distal end 16 may move towards the proximal end 14. As such, an axial direction of movement of the one or more protrusions 36 may be controlled by holding either the distal end 16 or the proximal end 14 of the valve body 12 in position during expansion. The protrusions 36 may be configured to move axially with the frame 20 in the direction of length contraction of the frame 20.

The prosthetic valve 10 may be deployed to a desired treatment site within a patient's body. The treatment site may be an implantation site for the prosthetic valve 10 to be implanted within the patient's body. The treatment site may be a valve of the patient's body, which may be a native valve, or may comprise a previously deployed prosthetic valve within the patient's body. The treatment site may include leaflets, which may be native leaflets (of a native valve) or may be host leaflets (of a previously deployed valve or host valve). The leaflets may suffer from a variety of maladies that may require the leaflets to be replaced with the leaflets of the prosthetic valve 10. For example, calcification of the leaflets or other conditions of the valve (e.g., stenosis or other conditions) may require the operation of the leaflets to be replaced with the leaflets of the prosthetic valve 10. The leaflets may remain within the patient's body upon the prosthetic valve 10 being deployed. The prosthetic valve 10 may be deployed in between the native or host leaflets such that the native or host leaflets are pushed radially outward upon expansion of the prosthetic valve. Such a configuration may result whether the prosthetic valve 10 is deployed within a native valve, or within a host valve that has been previously deployed.

The outward radial push of the native or host leaflets may cause a variety of maladies. For example, FIG. 4 illustrate a schematic view of a native aortic valve 48 including native valve leaflets 50 extending proximally, or in the outflow direction, from an annulus 52 of the aortic valve. The leaflets 50 are positioned between a flow channel of the native aortic valve 48 and a surface of the aorta (the ascending aorta). Notably, coronary ostia 54 may be positioned on the surface of the aorta. If a prosthetic valve were to be expanded within the native aortic valve 48, then there is a possibility that the expanded prosthetic valve may radially push the native leaflets 50 outward such that the native leaflets 50 may fully or partially cover one or more of the coronary ostia 54, which may lead to coronary maladies. A similar concern may result if a prosthetic valve were expanded within a previously expanded or host prosthetic valve within the aortic valve annulus 52.

The protrusions 36 may be utilized to displace one or more of the native leaflets 50 positioned outside of the flow channel 28 of the valve body 12 in a distal direction. The protrusions 36 extending outward from the outer surface 30 of the valve body 12 may contact the native leaflets 50 and displace the leaflets 50 distally to reduce the possibility of the leaflets 50 fully or partially covering the coronary ostia 54 or other coronary structures. Such an operation may occur for leaflets of a previously expanded or host prosthetic valve within the aortic valve annulus 52 if the prosthetic valve 10 (serving as a guest valve) were implanted in such a prosthetic valve.

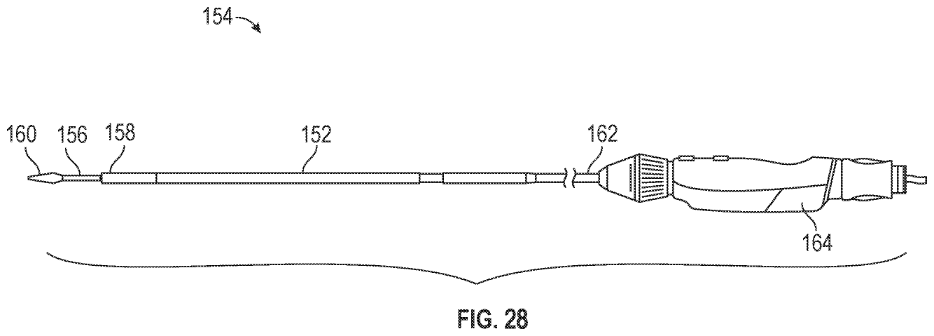

FIG. 4 illustrates the prosthetic valve 10 in a collapsed (or undeployed or unexpanded) state, in which the valve 10 may be configured to be expanded to increase the width 44 and decrease the length 18 (as marked in FIG. 3) of the valve body 12. The prosthetic valve 10 may be positioned upon an elongate shaft of a delivery apparatus (not shown in FIG. 4) that may be configured to deliver the valve 10 to the aortic valve annulus 52. An exemplary delivery apparatus 154 is shown in FIG. 28. The valve 10 may be passed by the elongate shaft in a transvascular manner, passing over the aortic arch to reach the aortic valve annulus 52. In other embodiments, other approaches may be utilized (e.g., transapical or another approach). In embodiments in which other native valves are treated, other approaches may be utilized to reach the native valves.

Referring to FIG. 4, upon the prosthetic valve 10 approaching the native aortic valve 48, the protrusions 36 may be positioned proximal of the proximal ends of the leaflets 50 (on the outflow side of the leaflets). The protrusions 36 may contact the proximal ends of the leaflets 50. The prosthetic valve 10 may then be expanded to increase the width 44 and decrease the length 18 (as marked in FIG. 3) of the valve body 12. An end, such as the distal end 16 of the prosthetic valve 10 may be held in an axial position (as marked by line 56) relative to the aortic valve annulus 52 or other portion of the native valve or other implantation site during expansion of the valve 10. The distal end 16 may be held in position by operation of the delivery apparatus that may be utilized to deploy the prosthetic valve 10. The distal end 16 being held in position may allow the proximal end 14 to move towards the distal end 16 and thus moving the one or more protrusions 36 distally.

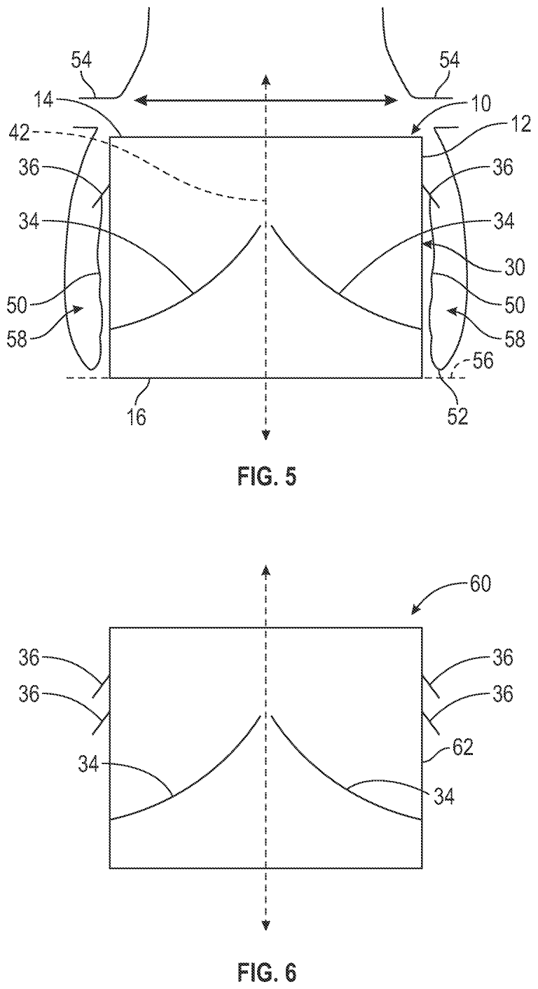

FIG. 5, for example, illustrates the prosthetic valve 10 in an expanded (or deployed) state. The prosthetic valve 10 is expanded within a patient's body. The length 18 of the valve body 12 has decreased and the width 44 (as marked in FIG. 3) of the valve body 12 has increased. The distal end 16 of the valve body 12 has been held in position, allowing the proximal end 14 to move towards the distal end 16 while the valve body 12 is radially expanded, thus moving the one or more protrusions 36 in a distal direction along with the movement of the frame 20. The one or more protrusions 36 contact and displace the native valve leaflets 50 in a distal direction, which may fold or otherwise compress the native valve leaflets 50. As such, the valve body 12 may be radially expanded to cause the protrusions 36 to displace the one or more leaflets. The proximal ends of the native valve leaflets 50 have been displaced in a direction distal of the coronary ostia 54. The leaflets 50 are retained distal of the coronary ostia 54 with the one or more protrusions 36, thus reducing the possibility of the native valve leaflets 50 fully or partially covering the coronary ostia 54 or other cardiac structures of the patient's body. The native valve leaflets 50 may have been displaced distal of the proximal ends 39 of the valve leaflets 34 (marked in FIG. 1). Upon implantation, the valve leaflets 34 operate as prosthetic replacements for the native valve leaflets 50.

The native valve leaflets 50 may be retained by the one or more protrusions 36 in a space 58 that is distal of the one or more protrusions 36 and is outside of the outer surface 30 of the valve body 12. The space 58 may be distal of the coronary ostia 54. The space 58 is bounded by the outer surface 30 of the valve body 12 and an external surface (here, the surface of the aorta) that surrounds the outer surface 30. The external surface may be an interior surface of the patient's vasculature such as the surface of the aorta, and/or may comprise a prior deployed prosthetic valve that surrounds the space 58, among other surfaces. The space 58 may be an annular space extending around the outer surface 30 of the valve body 12. Further, the angle of the protrusions 36 may serve to retain the native valve leaflets 50 against the outer surface 30 of the valve body 12, to prevent the leaflets 50 from moving radially outward from the outer surface 30. As such, the leaflets 50 may be held against the outer surface 30 of the valve body 12 to further reduce the possibility of the leaflets 50 fully or partially covering the coronary ostia 54.

The force of the outer surface 30 of the valve body 12 against the annulus 52 may anchor the prosthetic valve 10 within the annulus 52.

The configuration and operation of the prosthetic valve 10 may beneficially serve to reduce the possibility of obstruction of structures by native or host leaflets, and may utilize the expansion of the valve 10 to move the protrusions 36 in a desired direction to displace the native or host leaflets.

The configuration and operation of the prosthetic valve 10 may further allow for access to the structures with the valve 10 in position. For example, in an embodiment in which the valve 10 is deployed to the aortic valve, a space may be present between the outer surface 30 of the valve body 12 and the coronary ostia 54. The space may be proximal of one or more of the protrusions 36 and the native leaflets 50. The coronary ostia 54 may be accessed through the space. A catheter may be passed through the space to access the coronary ostia 54.

In embodiments, the native valve leaflets 50 may be displaced at least partially distal of one or more openings of the frame 20. Referring to FIG. 1, the frame 20 may include spaces 26 forming openings, and the protrusions 36 may displace the leaflets 50 at least partially distal of such an opening. The openings may be proximal of the proximal ends of the prosthetic valve leaflets 34. Fluid flow may thus be allowed through the opening to reach the coronary ostia 54. Further, a catheter may be passed through one of the openings from inside of the valve body 12 to outside of the valve body 12 to access the coronary ostia 54.

In embodiments, the prosthetic valve 10 may have a variety of forms, including a balloon expandable valve or a mechanically expandable valve as desired. Self-expanding valves may also be utilized. The delivery system utilized to deploy the valve 10 may be configured to hold the distal end 16 in position and expand the valve 10 according to the desired method of expansion (balloon expandable, mechanically expandable, self-expandable, among others).

Variations in the configuration and use of the prosthetic valve 10 may be provided.

FIG. 6 illustrates an embodiment of a prosthetic valve 60 configured similarly as the valve 10, yet including a plurality of protrusions 36 axially spaced from each other. The protrusions 36 may be positioned in one or more axially spaced levels relative to each other on the valve body 62. The protrusions 36 in each level may be circumferentially spaced from each other, such that the protrusions 36 form rows of protrusions extending around the outer surface of the valve body 62. A configuration of axially spaced protrusions 36 may increase the likelihood of the protrusions 36 contacting and displacing a leaflet upon expansion of the valve 60. For example, if a protrusion 36 on a lower (or distal) level fails to engage a leaflet, then a protrusion 36 on an upper (or proximal) level may engage the leaflet. Further, the axially spaced protrusions 36 may allow both levels of protrusions to engage the leaflet, by both contacting and displacing the leaflet. The configuration of protrusions 36 shown in FIG. 6 may be varied as desired.

FIG. 7 illustrates a frame 64 of a valve body in a flattened configuration. The frame 64 may be formed in the flattened configuration and then moved into a desired shape such as cylindrical (as shown in FIG. 1) or another shape as desired for use. The frame 64 may be cut in the flattened configuration from a flat plate of material, such that the frame 64 comprises a flattened body. For example, the frame 64 may be laser cut or otherwise formed from a flat plate of material to form the struts 66, junctures 68, and spaces 70 between struts 66 that form openings. The cut material may then be rolled to form a cylinder or other shape as desired. Certain of the spaces may comprise openings 67 for receiving the outer end portions 35 of the valve leaflets 34 and may serve as commissure points for the leaflets 34. The struts 66, junctures 68, and spaces 70 may comprise a pattern of cells that repeats circumferentially about the frame 64.

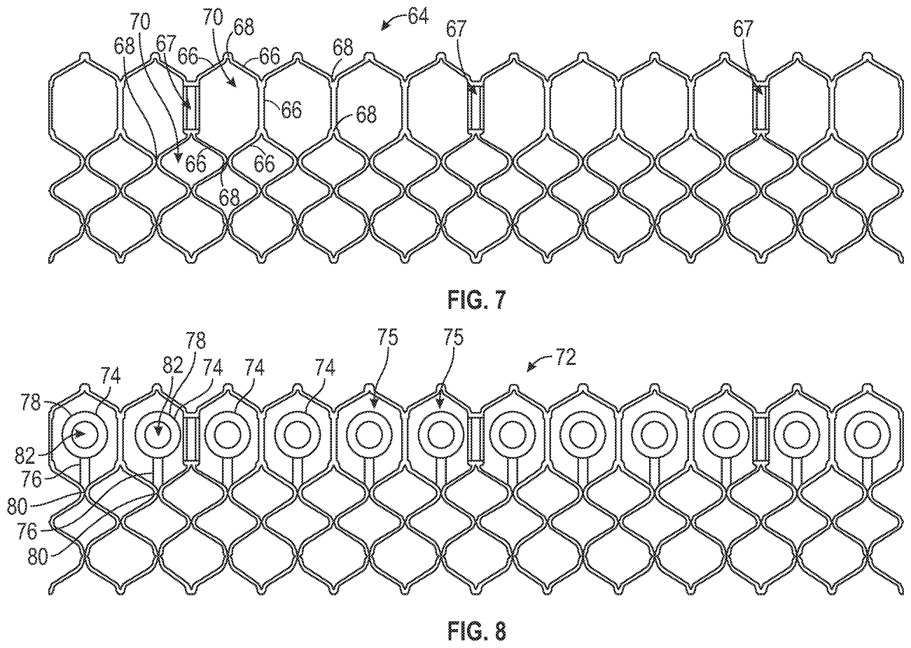

FIG. 8 illustrates a frame 72 formed to include one or more protrusions 74 integral with the frame 72. As such, as the pattern of the frame 72 is cut, the cut pattern may produce protrusions 74. The protrusions 74 may each comprise a flattened body formed from the same flattened body as the frame 72, and formed during a cutting process such as a laser cut or another form of cutting. The protrusions 74 may be formed to be initially positioned within an opening 75 of the frame 72, for example, a proximal or upper opening of the frame, configured similarly as the proximal or upper openings shown in FIG. 1.

The protrusions 74 may be formed each include a neck portion 76 and a head portion 78. The neck portion 76 may couple the head portion 78 to the frame 72, for example, to a juncture 80 of the frame 72. The juncture 80 may be positioned distal of the opening 75. The neck portion 76 may be formed initially extending proximally from the juncture 80. The neck portion 76 may be configured to be bendable to allow the neck portion 76 to be bent radially outward from the frame 72 as desired.

The head portion 78 may be formed and sized larger than the neck portion 76 and may be formed to be initially positioned in an opening 75. The head portion 78 may be a flattened body and may comprise an end of the protrusion 74. The head portion 78 may enclose an opening 82, such that the head portion 78 may comprise a ring of material extending around the opening 82. The opening 82 may reduce the amount of material comprising the head portion 78. The head portion 78 may have a rounded shape, such as circular or oval, or another shape as desired. The head portion 78 may have a diamond shape in embodiments, or a combination of shapes, or another shape as desired. The protrusions 74 may be formed in a pattern, repeating circumferentially about the frame 72. The neck portion 76 may comprise an end of the protrusion 74 coupled to the valve body and the head portion 78 may comprise an atraumatic tip of the protrusion 74.

Each protrusion 74 may be configured to be bent radially outward from the frame 72 such that the protrusion 74 extends outward from an outer surface of the valve body including the frame. Thus, after formation of the frame 72 and protrusions 74, the frame 72 may be formed into a desired shape such as cylindrical, and the protrusions 74 may be bent with the head portions 78 extending distally and the neck portions 76 forming a curved shape or “u” shape that bends the protrusions 74. The head portions 78 may extend distal of the openings 75.

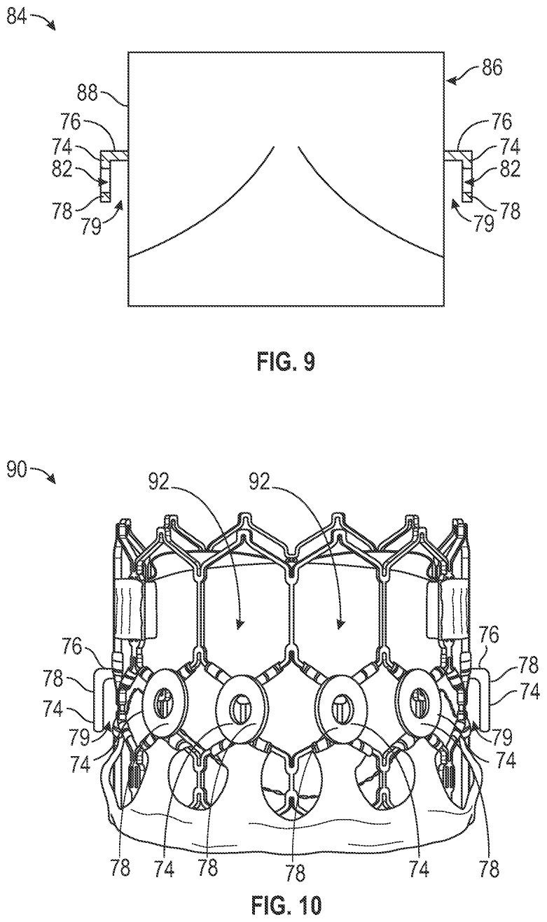

FIG. 9, for example, illustrates a cross sectional schematic view of a prosthetic valve 84 including the protrusions 74 extending outward from the outer surface 86 of the valve body 88, with the head portions 78 bent distally about the neck portions 76. The protrusions 74 are angled toward the distal direction. The head portions 78 may extend parallel with the outer surface 86 of the valve body 88 and may be oriented in a distal direction in embodiments, with the neck portion 76 extending radially outward. The neck portion 76 may extend radially outward perpendicular from the outer surface 86. Each protrusion 74 may be spaced from each other circumferentially about the outer surface 86 of the valve body 88.

A space 79 may be positioned between the outer surface 86 of the valve body 88 and the head portion 78, which may retain a native leaflet therein. The space 79 may be proximally bounded by the neck portion 76 and bounded radially outward by the head portion 78. The native leaflet accordingly may be displaced by the protrusion 74 and retained from extending radially outward from the outer surface 86 by being retained within the space 79. FIG. 10, for example, illustrates a perspective view of a prosthetic valve 90 including the protrusions 74.

The protrusions 74 may be configured to operate similarly as the one or more protrusions 36 discussed in regard to FIGS. 1-5. The one or more protrusions 74 may each be configured to extend outward from the outer surface of the valve body. The one or more protrusions 74 may be configured to displace one or more valve leaflets positioned outside of the flow channel of the valve body in a distal direction. The head portion 78 may serve as a large atraumatic tip for the protrusion 74 that may reduce the possibility of damage to a leaflet. The protrusions 74 may be configured to move according to the direction of expansion of the valve, as discussed in regard to FIGS. 1-5.

The axial position of the protrusions 74 may further be distal of openings 92 (marked in FIG. 10) of the frame, to allow fluid flow through the opening 92 and to a coronary ostia or other cardiac structure as desired. Access to the coronary ostia may also be provided through the openings via a catheter or another device as desired.

The shape of the head portion 78 may be varied in embodiments. FIG. 11 for example, illustrates an embodiment in which a head portion 94 has a diamond shape. The diamond shape may improve the crimp profile of the prosthetic valve utilizing the frame 96, and may improve the ability of the frame 96 to be crimped. The neck portions may be bent to orient the head portion in a similar manner as the embodiment of the protrusions shown in FIGS. 8-10.

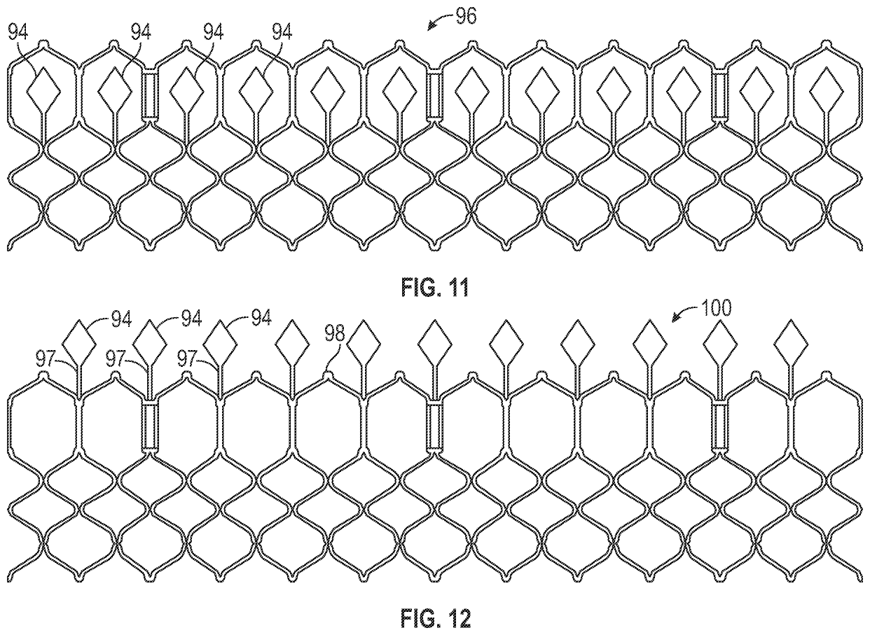

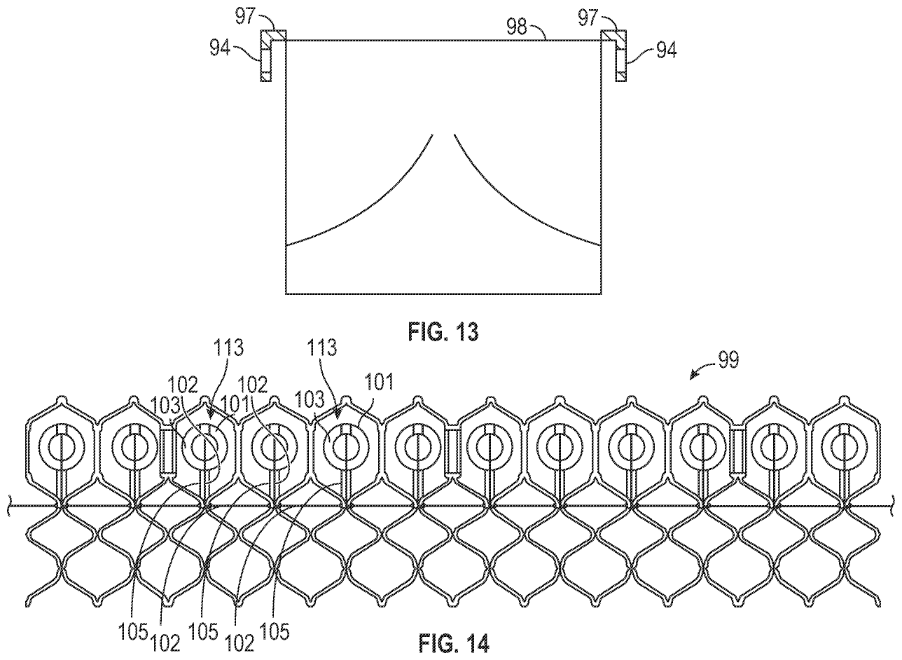

The position of the protrusions may be varied in embodiments. FIG. 12 for example illustrates that the protrusions 97 may be coupled to the proximal end 98 (or outflow end) of a frame 100. The protrusions 97 may then be bent distally from the proximal end 98 of the frame 100. FIG. 13 for example illustrates a cross sectional schematic view of such an embodiment, in which the protrusions 97 have a first end coupled to the proximal end 98 of the frame 100. The head portions 94 of the protrusions 97 comprise a tip of the protrusions 97 and extend distally from the proximal end 98 or outflow end of the frame 100. The neck portions may be bent to orient the head portion in a similar manner as the embodiment of the protrusions shown in FIGS. 8-10. Such a configuration may improve engagement with leaflets and may improve the crimp profile of the prosthetic valve.

In embodiments, the protrusions may be static relative to the valve body or frame to which they are coupled. In embodiments, the protrusions may be configured to move or rotate towards the distal direction. FIG. 14, for example, illustrates a configuration of a frame 99 that has the cut pattern of the frame 72 shown in FIG. 8. The frame 99 includes protrusions 101 having a head portion 103 configured similarly as the head portion 78 shown in FIG. 8. The neck portion 105, however, may comprise a hinge that the head portion 103 may rotate about towards the distal direction.

One or more tethers 102 may be coupled to the protrusions 101 and may be coupled to the frame 99. Each tether 102 may couple the valve body to a respective one of the one or more protrusions 101 and may be configured to apply a force to the respective one of the one or more protrusions 101 to rotate the respective one of the one or more protrusions 101 towards the distal direction. The frame 99 may be an expandable frame and the one or more tethers 102 may be coupled to the expandable frame such that expansion of the frame causes the one or more tethers 102 to apply a force to the respective one of the one or more protrusions 101.

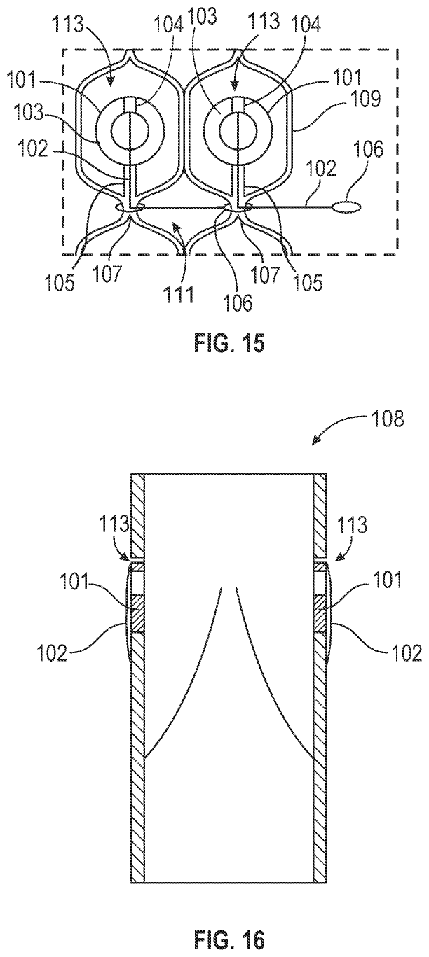

FIG. 15 illustrates a close up view of a portion of the frame 99 showing a configuration of the tethers 102. Each tether 102 may include a first end 104 or proximal end coupled to a respective one of the protrusions 101. The first end 104 may couple to the head portion 103 of the protrusions 101. The coupling may occur towards the tip of the head portion 103 or may be in another location as desired (further distal on the head portion 103 as desired, or the coupling may occur on the neck portion 105). The first end 104 may loop around the body of the respective protrusion 101. Each tether 102 may include a second end 106 that couples to a portion of the frame 99. The portion of the frame 99 may comprise struts 109 of the frame, which are separated by the openings of the frame 99. For example, the coupling may occur at a juncture 107 of the struts 109 of the frame 99. The coupling may occur at a juncture that is circumferentially offset from the location of the respective protrusion 101. Each end 106 may comprise a loop or other configuration.

A looped configuration may allow the body of another adjacent tether 102 to pass through the looped end 106. The body of the tether 102 may then span the spaces between the junctures 107. The body of the tether 102 may extend from the looped end 106 circumferentially leftward as shown in FIG. 15, and then may pass through the looped end of an adjacent tether. The body of the tether 102 may then deflect upward in the proximal direction to couple to the head portion 103. As such, as the frame 99 is expanded radially outward, the width of the spaces 111 between the junctures 107 increases. The looped end 106 is thus pulled circumferentially, which pulls the body of the tether 102 circumferentially. Due to the deflection of the tether 102 through an adjacent loop, the portion of the tether 102 coupled to the head portion 103 is pulled distally. The protrusion 101 is accordingly pulled distally and may rotate distally about the neck portion 105. The tethers 102 may accordingly expand the protrusions 101 radially outward. The configuration of the one or more tethers 102 may be varied in embodiments, for example a single loop may extend around the entire outer surface of the frame such that expansion of the valve body causes the loop to expand and pull on the protrusions 101 distally.

FIG. 16, for example, illustrates a cross sectional schematic view of the embodiment shown in FIGS. 14 and 15. FIG. 16 illustrates a prosthetic valve 108 having the protrusions 101 configured to rotate in a distal direction. The prosthetic valve 108 is shown in a collapsed (or undeployed or unexpanded) state. The protrusions 101 may be positioned within the openings 113 of the frame 99 marked in FIG. 14 and may extend in a proximal direction. In such a configuration, the outer diameter of the prosthetic valve 108 is not increased by the presence of the protrusions 101 when in the collapsed configuration. As such, the valve 108 may have a relatively low profile or diameter with the protrusions 101 extending proximally, which may be same diameter or profile as if the protrusions 101 were not present. Such a feature may improve the ability of the valve 108 to be delivered to a treatment site while maintaining a relatively low outer diameter. The protrusions 101 may have an outer diameter that is less than or no greater than the outer diameter of the outer surface of the valve body. As discussed, upon the prosthetic valve 108 being expanded, the tethers 102 may pull and rotate the protrusions 101 towards the distal direction.

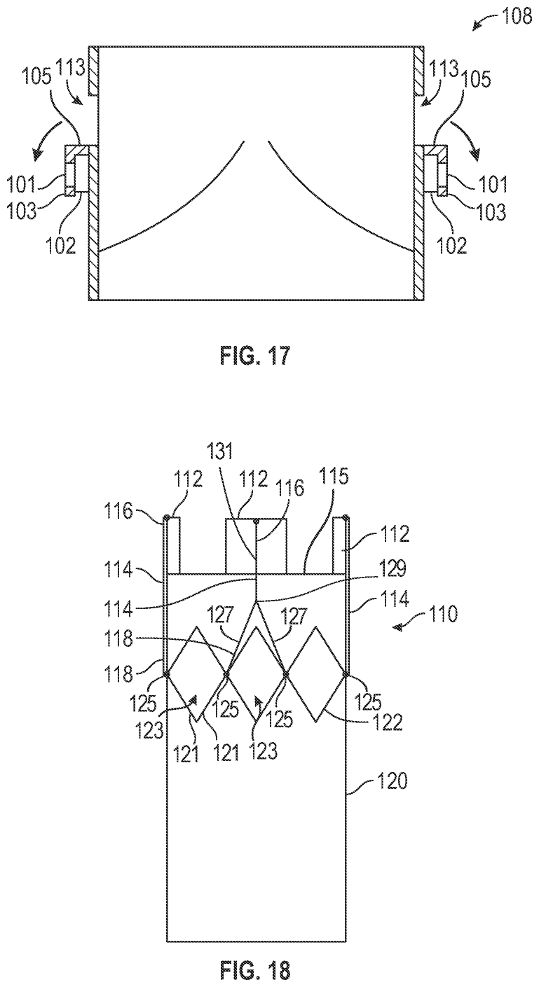

FIG. 17, for example, illustrates the protrusions 101 having been rotated distally by a pulling force applied by the tethers 102. The tethers 102 are utilized to apply a force to the protrusions 101 to rotate the protrusions towards the distal direction. The head portions 103 rotate distally and are oriented distally, parallel with the outer surface of the valve body. The head portions 103 rotate about the neck portions 105, which may comprise hinges. The protrusions 101 may be in an orientation as shown in FIG. 9 for example, and may operate in a similar manner as the protrusions shown in FIG. 9. The pulling force is caused by the radial expansion of the prosthetic valve 108. In such a configuration, the proximal end or distal end of the valve body may need not be held in a particular position during expansion, as the distal movement of the protrusions 101 due to the rotation about the neck portions 105 may displace the leaflets distally. Such a feature may be used solely to displace the leaflets distally, or may be utilized in combination with an expansion of the valve causing distal movement of the protrusions as disclosed herein.

The one or more protrusions 101 may each be configured to extend outward from the outer surface of the valve body. The one or more protrusions 101 may be configured to displace one or more valve leaflets positioned outside of the flow channel of the valve body in a distal direction.

Other configurations of the protrusions and mechanisms for rotating or deploying the protrusions may be utilized.

FIG. 18 illustrates an embodiment of a prosthetic valve 110, in which the protrusions comprise one or more flaps 112 that are each configured to rotate towards the distal direction. The flaps 112 may be coupled to the valve body 120, and may be positioned at the proximal end 115 of the prosthetic valve 110. The flaps 112 may be coupled to the proximal end 115 of the valve body with a hinge 117 (as marked in FIG. 19). An opposite end of the flap 112 may comprise an atraumatic tip of the flap 112. The flaps 112 may each be circumferentially spaced from each other about the outer surface of the valve body 120. Three flaps 112, or a greater or lesser number of flaps 112 may be utilized as desired.

The flaps 112 may each be configured as bodies having a width and a length and forming a wide surface area for contacting and engaging leaflets. The flaps 112 may be configured to form an arcuate shape, as shown in FIG. 19, upon being rotated in the distal direction. Other shapes may be utilized for the flaps 112 as desired. Other positions of the flaps 112 may be utilized as well. Any of the embodiments of protrusions disclosed herein may include flaps.

FIG. 18 illustrates the prosthetic valve 110 in a collapsed (or undeployed or unexpanded) state. The flaps 112 in such a configuration may extend in a proximal direction, and may extend parallel with the outer surface of the valve body 120. The flaps 112 may protrude proximally from the proximal end 115 of the valve body 120 but do not protrude radially outward. In such a configuration, the outer diameter of the prosthetic valve 110 may not be increased by the presence of the flaps 112 when in the collapsed configuration. As such, the valve 110 may have a relatively low profile or diameter with the flaps 112 extending proximally, which may be same diameter or profile as if the flaps 112 were not present. Such a feature may improve the ability of the valve 110 to be delivered to a treatment site while maintaining a relatively low outer diameter. The flaps 112 may have an outer diameter that is less than or no greater than the outer diameter of the outer surface of the valve body 120.

Each flap 112 may be coupled to a tether 114 that may operate similarly as the tether 102 discussed in regard to FIGS. 14-17. Each tether 114 may couple the valve body 120 to a respective one of the one or more protrusions in the form of flaps 112 and may be configured to apply a force to the respective one of the one or more flaps 112 to rotate the flaps 112 towards the distal direction. The valve body 120 may include an expandable frame, and the one or more tethers 114 may be coupled to the expandable frame such that expansion of the frame causes the one or more tethers 114 to apply a force to the flaps 112. Each tether 114 may have a first end 116 coupled to a respective flap 112 and may have an opposite second end 118 coupled to a portion of the valve body 120 that may comprise a frame 122. A portion of the frame 122 is shown in FIG. 18, to include struts 121 separated by openings 123, and connected at junctures 125.

Each tether 114 may be coupled to the frame 122 such that radial expansion of the frame 122 causes the tether 114 to apply a force to the respective flap 112 causing the respective flap to rotate distally. The tethers 114 are utilized to apply a force to the flaps 112 to rotate the flaps towards the distal direction. The second end 118 of each tether, for example, may couple to the junctures 125 of the struts 121 of the frame 122, and as the width of the space (e.g. openings 123) between the junctures increases, the tether 114 may pull on the respective flap 112 distally. The tether 114 may have a “Y” shape as shown in FIG. 18, with two distal bodies 127 extending outward from a juncture 129 of the tether 114. A proximal body 131 may extend upward from the juncture 129.

FIG. 19 illustrates the frame 122 having been expanded, thus causing the flaps 112 to rotate distally. The flaps 112 may fold over the proximal edge of the prosthetic valve. The tether 114 may move from a “Y” shape as shown in FIG. 18 to a “T” shape as shown in FIG. 19. The pull of the tethers 114 may rotate the flaps 112 about the respective hinges 117. The flaps 112 accordingly may extend radially outward from the outer surface of the valve 110 and may be configured to displace one or more valve leaflets positioned outside of the flow channel of the valve body in the distal direction. The flaps 112 are angled toward the distal direction. The protrusions in the form of the flaps 112 may displace the one or more valve leaflets in a similar manner as disclosed herein. The rotation of the flaps 112, however, may allow the proximal end or distal end of the valve body to not be fixed in a particular position during expansion, as the distal movement of the flaps 112 may displace the leaflets distally. Such a feature may be used solely to displace the leaflets distally, or may be utilized in combination with an expansion of the valve causing distal movement of the protrusions as disclosed herein.

The wide surface area of the flaps 112 may increase the likelihood of the flaps 112 engaging the leaflets and displacing the leaflets distally.

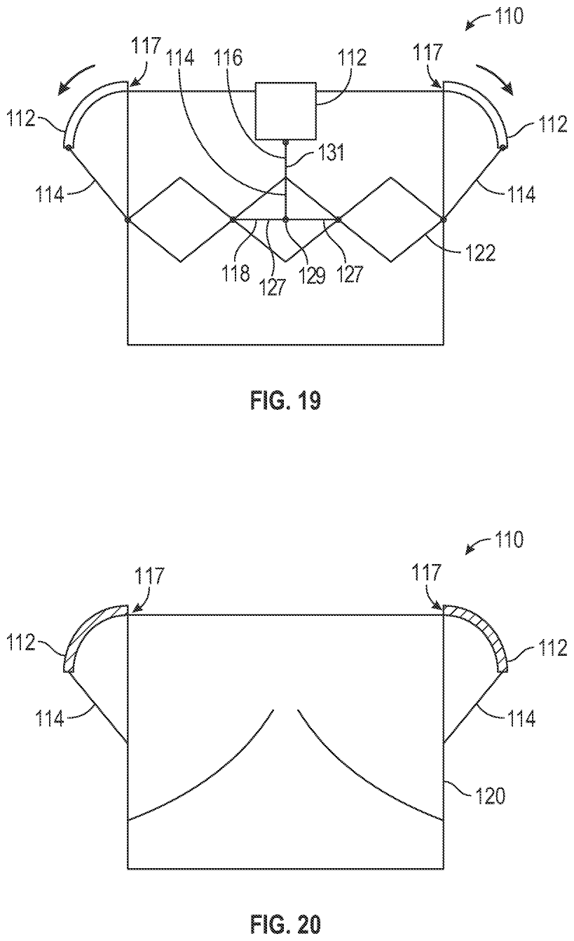

FIG. 20 illustrates a cross sectional schematic view of the prosthetic valve 110 in the expanded configuration.

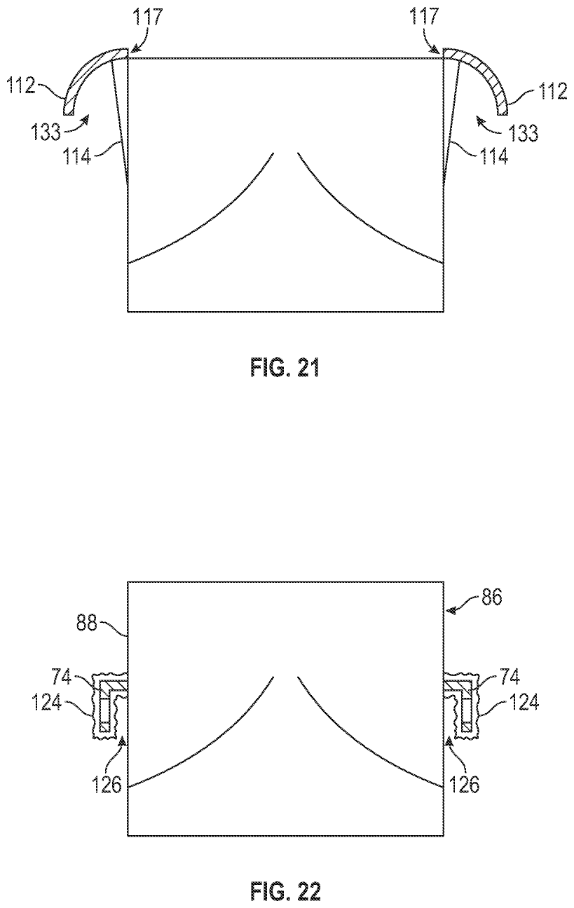

The tethers 114 may couple to the valve body 120 in a variety of locations and may couple to a respective one of the flaps 112 in a variety of locations as well. For example, as shown in FIG. 21, a tether 114 may couple proximate one of the hinges 117 in certain embodiments (or distally from the tips of the flaps). This is in contrast with an embodiment shown in FIG. 20, in which the tether 114 may couple to the flap 112 proximate the tips of the flaps 112 and distally from the hinges 117. In a configuration as shown in FIG. 21, a greater surface area of the flap 112 may be available to engage and displace the leaflets because the tether 114 is coupled further from the tip of the flap 112 than shown in FIG. 20. A space 133 may be provided between the flaps 112 and the outer surface of the valve body to retain the leaflets therein.

The protrusions, in various forms disclosed herein, may comprise one protrusion in certain embodiments. For example, in an embodiment in which the protrusions comprise flaps, one flap may extend from the valve body and be configured to displace the leaflets as desired.

FIG. 22 illustrates a side cross sectional schematic view of an embodiment in which a skirt 124 couples to and covers one or more protrusions 74. The skirt 124 may be made of a material having openings that allow fluid to pass through, yet are appropriately sized to allow the skirt 124 to capture material such as emboli. The skirt 124 may be made of a woven material and may be made of a polymer or other material in embodiments. The skirt 124 may serve as a filter for filtering fluid (such as blood) passing through the skirt 124.

The skirt 124 may extend circumferentially around the entirety of the outer surface of the valve body 88, or over a portion thereof. The skirt 124 may extend radially outward from the outer surface 86 of the prosthetic valve 84 and may cover one or more protrusions 74 extending from the outer surface 86. The protrusions 74 may be configured similarly as the protrusions 74 shown in FIG. 9, or may be configured to rotate in certain embodiments or have another configuration.

The skirt 124 may form a space 126 between the skirt 124 and the outer surface of the valve body 88. The space 126 may be annular in shape around the outer surface of the valve body. The space 126 may be configured to capture material that may pass through the patient's vasculature. Such material may comprise emboli or other material that may be dislodged during the implantation process. The skirt 124 may capture such material to reduce the possibility of maladies associated with transmission of such materials, such as stroke or the like. Such material may be captured during deployment of the prosthetic valve, or before or after such deployment. The skirt 124, for example, may capture material produced due to the displacement of the leaflets by the protrusions 74 covered by the skirt 124. Such material (such as calcified material) may be released from the leaflet during displacement of the leaflet.

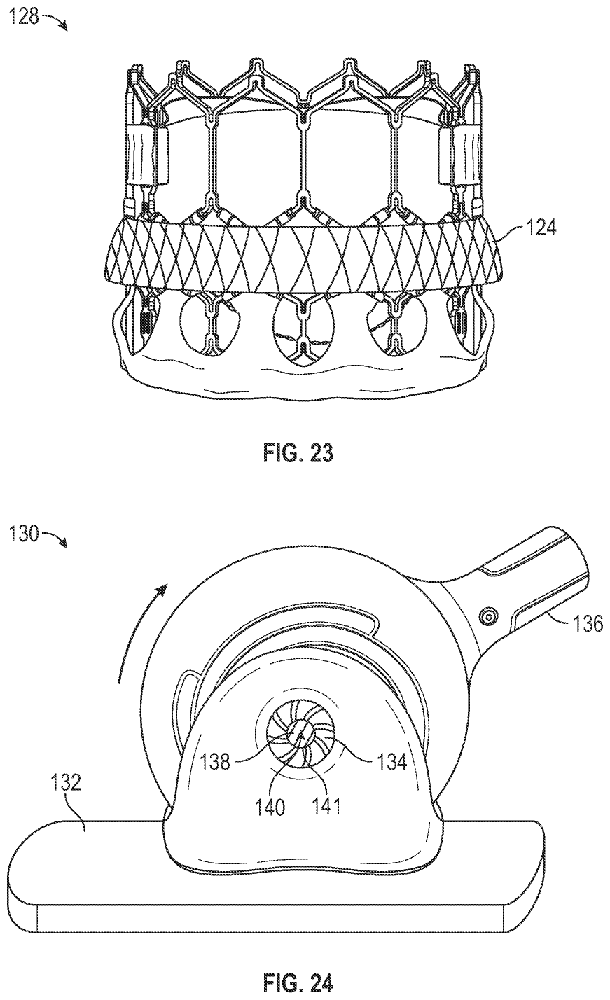

The protrusions 74 covered by the skirt 124 may continue to serve to displace valve leaflets in a distal direction. FIG. 23, for example, illustrates a perspective view of such a skirt 124 covering protrusions 74 (as shown in FIG. 22) and extending radially outward from the outer surface of a valve body of a prosthetic valve 128.

In an embodiment in which a prosthetic valve includes protrusions as disclosed herein, a crimping device may be utilized to crimp the prosthetic valve and accommodate the protrusions that may extend outward from the valve body. FIG. 24, for example, illustrates such a crimping device 130. The crimping device 130 may include a base 132, a compressive body 134, and an actuator 136 configured to operate crimping device by contracting an inner surface of the crimping device.

The base 132 may be configured to be positioned upon a surface, and may support the compressive body 134 and the actuator 136. The actuator 136 may comprise a handle that is configured to be rotated or may have another configuration as desired. For example, in embodiments the actuator 136 may comprise a pump, a motor, or other mechanism for actuating the crimping device. In an embodiment in which the actuator 136 is a handle, the handle may rotate to move the compressive body 134 to have a reduced inner diameter.

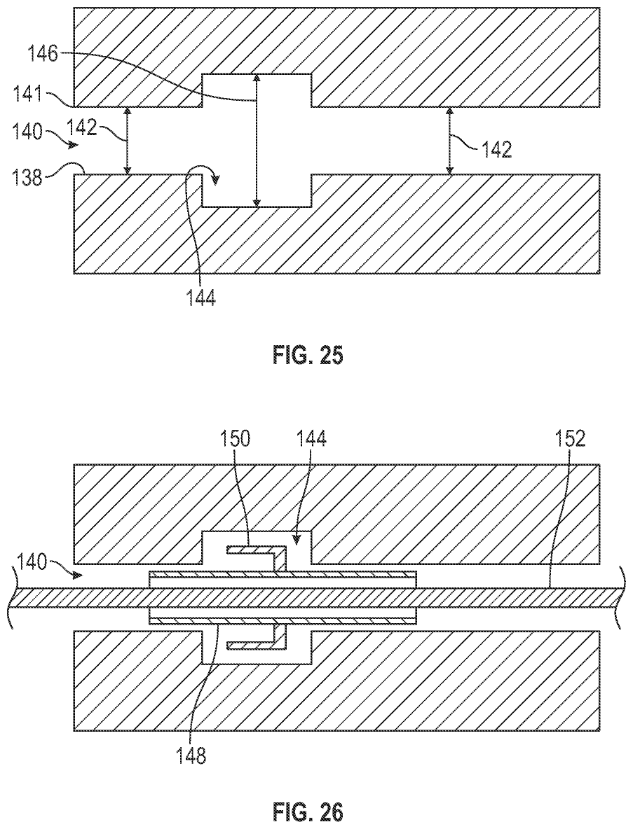

The compressive body 134 may have an inner surface 138 that surrounds a channel 140 that is configured to receive a prosthetic valve. The valve may be passed through an opening 141 to enter the channel 140. The inner surface 138 may be configured to be contracted to apply a compressive force to the prosthetic valve within the channel 140 to crimp the prosthetic valve. The compressive body 134 may be configured in a variety of manners, including a plurality of plates, forming an iris structure, in which rotation of the plates causes an interior diameter 142, 146 of the channel 140 to reduce (as marked in FIG. 25). The inner surface 138 accordingly contracts upon rotation of the plates. The actuator in the form of a handle may be rotated to cause the plates to move to reduce the interior diameter. In other embodiments, other configurations of compressive bodies may be utilized, including levers and bladders, among others.

FIG. 25 illustrates a cross sectional schematic view of the channel 140 configured to receive the prosthetic valve. The channel 140 may be defined and surrounded by the inner surface 138 that is configured to contract. The inner surface 138 may have an interior diameter 142. The inner surface 138 may have a recess 144 shaped in the inner surface 138 that may accommodate a protrusion of the prosthetic valve. The recess 144 may have an interior diameter 146 that is larger than the interior diameter 142 of the inner surface 138 outside of the recess 144. The interior diameter 146 of the recess 144 and the interior diameter 146 of the inner surface 138 outside of the recess 144 may each be uniform. The recess 144 may have a cylindrical shape, and the channel 140 outside of the recess 144 may have a cylindrical shape as well. The recess 144 may comprise a groove formed in the inner surface 138.

FIG. 26 illustrates a prosthetic valve 148 in an uncrimped or expanded state including protrusions 150, which may be configured similarly as the protrusions disclosed herein. The prosthetic valve 148 may be positioned in the channel 140 and positioned upon an elongate shaft 152 of a delivery apparatus that the prosthetic valve 148 is to be crimped to. FIG. 28, for example, illustrates a delivery apparatus 154 that the prosthetic valve 148 may be crimped to, and may be utilized to deliver any of the prosthetic valves disclosed herein to a desired implantation site. The configuration of the delivery apparatus 153 may be varied in other embodiments. The delivery apparatus 154 may include the elongate shaft 152, which may include an implant retention area 156 that the valve 148 is crimped to. A sheath of the elongate shaft 152 may cover the crimped or compressed, or undeployed valve 148 in the implant retention area 156 to retain the valve 148 to the shaft 152. The implant retention area 156 may be positioned at a distal end 158 of the elongate shaft 152. A nosecone 160 may further be positioned at the distal end 158 of the elongate shaft 152. The elongate shaft 152 may include a proximal end 162 that may couple to a handle 164 utilized to grip the delivery apparatus 154, or may couple to another form of housing.

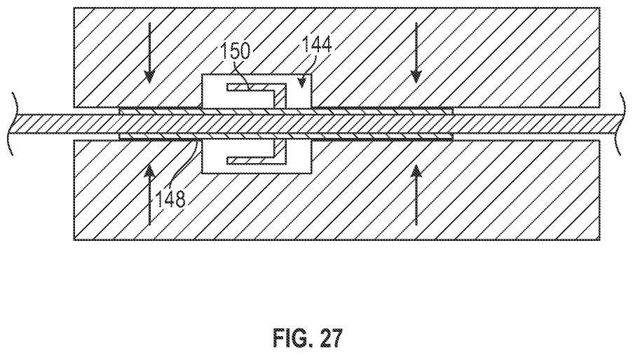

Referring back for FIG. 26, the protrusions 150 may be positioned within the recess 144. The inner surface 138 may be compressed against the prosthetic valve 148 with a radially compressive force to crimp the prosthetic valve 148. FIG. 27, for example, illustrates the compressive force applied to the prosthetic valve 148. The prosthetic valve 148 is crimped with the protrusions 150 continuing to extend radially outward from the outer surface of the valve body. The recess 144 may prevent the protrusions 150 from being compressed to the same diameter as the rest of the valve 148. A portion of the inner surface 138 within the recess 144 may contact the protrusions 150 during crimping, or may be spaced from the protrusions 150 during crimping as shown in FIG. 27. The valve 148 may be crimped to the elongate shaft 152, at the implant retention area 156 shown in FIG. 28 for example. The valve 148 may be coupled to the delivery apparatus and in position to be deployed to the desired treatment site.

In certain embodiments, a cushioning material may be applied to the prosthetic valve 148. The cushioning material may comprise Qualcrimp® or another form of cushioning material as desired. Such cushioning material may cushion the valve and the protrusions 150 during crimping to avoid damage to the protrusions during crimping.

The embodiments as disclosed herein may be discussed in regard to a prosthetic valve, however, the systems, devices, and methods disclosed herein are not limited to prosthetic valves. Other forms of implants and prosthetic implants may utilize the systems, devices, and methods disclosed herein, including stents and other forms of medical implants.

The systems, devices, and methods disclosed herein are not limited to treatment of the aortic valve, but may extend to mitral, pulmonary, and tricuspid valves, as well as treatment of other portions of a patient's body. The systems, devices, and methods disclosed herein may be utilized as docking support members that may be folded over and seated against native anatomical structures, such as a native mitral valve or other location. Other uses may be provided. The embodiments of protrusions may be utilized to displace leaflets or may be configured to displace other anatomical structures as desired in embodiments.

The implants may be cylindrical implants, or in other embodiments may have other shapes such as “V” shaped implants or other shapes as desired. The implants may be configured to expand radially outward from an axis that the implant surrounds, for example a longitudinal axis of the implant. The implants may be balloon expandable, mechanically expandable, or may be self-expanding in embodiments. The delivery apparatuses utilized, for example, may be configured to produce the desired form of expansion. For a balloon expandable valve, for example, the delivery apparatus may include an expansion balloon and may include a lumen for inflating and expanding the balloon positioned interior of the valve. For a mechanically expandable valve, the delivery apparatus may include a mechanical deployment mechanism for expanding the valve. For a self-expanding valve, the delivery apparatus may include a retractable sheath or the like for uncovering the valve and allowing the valve to expand. Other forms of deployment and delivery apparatuses may be utilized as desired.

The systems, devices, and methods disclosed herein may be used in a variety of procedures, which may include transcatheter aortic valve implantation (TAVI). The delivery apparatus and the systems disclosed herein may be utilized for transarterial access, including transfemoral access, to a patient's heart. The approach to the delivery site may be in a variety of manners. For example, an approach to a native aortic valve may be through an aortic arch. In embodiments, a ventricular approach may be utilized, approaching the native aortic valve from the inflow side of the native aortic valve. A prosthetic valve may be implanted from such a direction, with protrusions displacing native leaflets distally.

In embodiments, the systems, devices, and method disclosed herein may be utilized for mitral, tricuspid, and pulmonary replacement and repair as well. The delivery systems may be utilized in transcatheter percutaneous procedures, including transarterial procedures, which may be transfemoral or transjugular. Transapical procedures, among others, may also be utilized. The systems, devices, and methods disclosed herein may be utilized to deploy a guest prosthetic valve within a host prosthetic valve to treat a failure of operation of the host prosthetic valve. Such embodiments may comprise Valve in Valve (ViV) procedures.

The tethers as disclosed herein may comprise flexible bodies such as sutures, cords, cables, or wires, or may comprise other forms of tethers as desired.

Features of embodiments may be modified, substituted, excluded, or combined across embodiments as desired.

In addition, the methods herein are not limited to the methods specifically described, and may include methods of utilizing the systems and devices disclosed herein. The steps of the methods may be modified, excluded, or added to, with systems, devices, and methods disclosed herein.

The features of the embodiments disclosed herein may be implemented independently of other components disclosed herein. The various apparatuses of the systems may be implemented independently.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of systems, apparatuses, and methods as disclosed herein, which is defined solely by the claims. Accordingly, the systems, apparatuses, and methods are not limited to that precisely as shown and described.

Certain embodiments of systems, apparatuses, and methods are described herein, including the best mode known to the inventors for carrying out the same. Of course, variations on these described embodiments will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described embodiments in all possible variations thereof is encompassed by the systems, apparatuses, and methods unless otherwise indicated herein or otherwise clearly contradicted by context.

Groupings of alternative embodiments, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A prosthetic valve comprising:
a valve body having a proximal end, a distal end, an outer surface, and an inner surface facing a flow channel;
a plurality of valve leaflets positioned within the flow channel and extending inward from the inner surface of the valve body; and
one or more protrusions configured to extend outward from the outer surface of the valve body and configured to displace one or more valve leaflets positioned outside of the flow channel of the valve body in a distal direction.

2. The prosthetic valve of claim 1, wherein the one or more protrusions are configured to retain the one or more valve leaflets in a space distal of the one or more protrusions and outside of the outer surface of the valve body.

3. The prosthetic valve of claim 1, wherein the one or more protrusions each include a first end coupled to the valve body and a second end having an atraumatic tip.

4. The prosthetic valve of claim 3, wherein the second end includes a flattened body.

5. The prosthetic valve of claim 1, further comprising one or more tethers coupling the valve body to a respective one of the one or more protrusions, and configured to apply a force to the respective one of the one or more protrusions to rotate the respective one of the one or more protrusions towards the distal direction.

6. The prosthetic valve of claim 5, wherein the valve body includes an expandable frame, and the one or more tethers are coupled to the expandable frame such that expansion of the expandable frame causes the one or more tethers to apply the force to the respective one of the one or more protrusions.

7. The prosthetic valve of claim 6, wherein the expandable frame has struts separated by openings, and each of the one or more tethers has a first end coupled to the struts and a second end coupled to the respective one of the one or more protrusions.

8. A method comprising:
expanding a prosthetic valve within a patient's body, the prosthetic valve including a valve body having a proximal end, a distal end, an outer surface, and an inner surface facing a flow channel, and a plurality of valve leaflets positioned within the flow channel and extending inward from the inner surface of the valve body; and
displacing one or more valve leaflets positioned outside of the flow channel of the valve body in a distal direction with one or more protrusions extending outward from the outer surface of the valve body.

9. The method of claim 8, further comprising retaining the one or more valve leaflets positioned outside of the flow channel in a space distal of the one or more protrusions and outside of the outer surface of the valve body.

10. The method of claim 9, wherein an interior surface of a patient's vasculature surrounds the space.

11. The method of claim 10, wherein the interior surface is a surface of the patient's aorta.

12. The method of claim 9, wherein a prior deployed prosthetic valve surrounds the space.

13. The method of claim 8, further comprising utilizing one or more tethers to apply a force to a respective one of the one or more protrusions to rotate the respective one of the one or more protrusions.

14. The method of claim 13, wherein the one or more tethers are coupled to a frame of the valve body.

15. The method of claim 14, further comprising radially expanding the frame to cause the one or more tethers to apply the force to the respective one of the one or more protrusions.

16. The method of claim 8, further comprising radially expanding the valve body to cause the one or more protrusions to displace the one or more valve leaflets positioned outside of the flow channel of the valve body in the distal direction.

17. The method of claim 16, further comprising moving the proximal end of the valve body towards the distal end of the valve body while radially expanding the valve body.

18. The method of claim 8, wherein the valve body includes a frame having one or more openings, and the method comprises passing a catheter through one of the one or more openings to access one or more coronary ostia.

19. A crimping device for a prosthetic valve, the crimping device including:
a compressive body having an inner surface surrounding a channel configured to receive the prosthetic valve, the inner surface configured to be contracted to apply a compressive force to the prosthetic valve within the channel to crimp the prosthetic valve, the inner surface having a recess shaped in the inner surface to accommodate a protrusion of the prosthetic valve; and
an actuator for contracting the inner surface.

20. The crimping device of claim 19, wherein the inner surface outside of the recess has an interior diameter that is uniform.
